(19) United States
(12) Patent Application Publication
(10) Pub. No.: US 2025/0037839 A1
(43) Pub. Date: Jan. 30, 2025

(54) SYSTEMS AND METHODS FOR PREDICTING MENTAL HEALTH CRISIS SCENARIOS AND INITIATING INTERVENTION

(71) Applicant: Wellness Empowered, LLC, San Antonio, TX (US)

(72) Inventors: Gerard Berlanga, San Antonio, TX (US); Antonio Santana, San Antonio, TX (US); Carl Burnside, Cedar Creek, TX (US)

(73) Assignee: Wellness Empowered, LLC, San Antonio, TX (US)

(21) Appl. No.: 18/911,894

(22) Filed: Oct. 10, 2024

Related U.S. Application Data
(63) Continuation-in-part of application No. 18/749,124, filed on Jun. 20, 2024.
(60) Provisional application No. 63/522,072, filed on Jun. 20, 2023.

(51) Int. Cl. G16H 20/70; G16H 50/30; G16H 80/00

(52) U.S. Cl. CPC G16H 20/70; G16H 50/30; G16H 80/00

(57) ABSTRACT

A software-based system for mental health management and suicide prevention that utilizes biometric data from wearable devices. The system extracts and analyzes physiological metrics, such as heart rate, sleep patterns, and physical activity levels, to identify patterns, trends, or anomalies indicative of emerging mental health issues. Advanced algorithms process the collected data to detect potential mental health concerns before they escalate into crises, enabling proactive intervention and support.

### CROSS-REFERENCE TO RELATED APPLICATIONS

This application is a continuation-in-part of U.S. patent application Ser. No. 18/749,124, filed Jun. 20, 2024, titled “SYSTEMS AND METHODS FOR PREDICTING CRISIS SCENARIOS AND INITIATING SUICIDE INTERVENTION,” which claims the benefit of U.S. Provisional Application 63/522,072, filed Jun. 20, 2023, titled “SYSTEMS AND METHODS FOR PREDICTING CRISIS SCENARIOS AND INITIATING SUICIDE INTERVENTION. The contents of the above identified applications are incorporated herein by reference in their entirety.

### BACKGROUND

### Field of the Art

The invention relates to predicting mental health crisis scenarios.

### Discussion of the State of the Art

Existing suicide prevention methods are typically reactive, being put into action after a situation has occurred where an individual was at risk of suicide. These methods often depend on measures or indicators that are lagging, such as self-reported thoughts, feelings, emotions, and lifestyle choices. These are usually discussed with a medical professional after the situation associated with potential self-harm or suicide has occurred. While this method allows for measures to be established to try to avoid future dangerous situations, it offers limited monitoring of patient well-being where an effective intervention could be implemented. These methods are not proactive or predictive and generally do not allow for intervention at the moment where prevention of self-harm or suicidal actions could be implemented.

This method of treating at-risk patients also depends on the patient accurately recording or journaling mood and lifestyle information over time for later review by a clinician. It generally lacks additional data, such as heart rate, pulse, sleep, etc., which could be useful in identifying at-risk situations. This method also requires an individual to recall something that may have happened several weeks ago, which can be difficult. This makes it hard for a therapist to address the situation and provide appropriate treatment or change in treatment.

Conventional methods generally do not provide ongoing real-time feedback to users about how certain lifestyle choices are directly impacting their mood and/or mental health. The current treatment model also fails to provide timely information that would allow a patient to learn and correct certain behaviors that have a negative impact on their mood. The current treatment model is reactive in nature. The current methods also place the burden on the patient to recognize adverse circumstances, which may be difficult for an individual to recognize and/or the individual may not be capable of an ideal response when they arise.

In addressing the escalating issue of mental health, particularly in the pervasive challenge of suicide prevention, current methods primarily exhibit a reactive nature, which greatly limits their efficacy. Traditional approaches in mental health interventions rely heavily on self-reported data from patients during their interactions with mental health professionals. This inherently subjective mechanism of gathering information has many limitations, notably the potential for inaccurate or incomplete reporting by patients, either due to recall bias or the stigma associated with mental health issues, which may deter individuals from fully disclosing their mental state.

Moreover, the conventional strategies employed for suicide prevention and mental health management have predominantly been centered on addressing symptoms and crises as they arise, rather than implementing preemptive measures. This reactive model results in missed opportunities for intervention before individuals reach a level of mental health crisis. This underscores the need for a more anticipatory approach to mental health care that can identify and mitigate risks before they escalate into emergencies.

### SUMMARY

The present invention is a software-based solution that uses biometric data from wearable devices to facilitate a proactive approach to mental health management and suicide prevention. The system extracts and analyzes a wide array of physiological metrics collected by wearable technology, such as heart rate, sleep patterns, physical activity levels, and other indicators that may correlate with the mental state of an individual. This information is synthesized and processed using advanced algorithms to identify patterns, trends, or anomalies that could indicate emerging mental health issues before they evolve into crises.

This invention provides detailed, objective data to therapists and peer support groups, enabling these professionals to gain a more nuanced understanding of a patient's well-being. This insight enriches the therapist-patient dialogue and empowers mental health practitioners to customize treatment plans more effectively. Unlike existing approaches, which are predominantly reactive and heavily reliant on subjective patient self-reporting, the invention introduces a method of continuous, objective monitoring. This proactive model enhances the capability to intervene at an earlier stage, potentially averting mental health crises or episodes of suicidal ideation.

The invention improves upon prior solutions by integrating biometric data analysis with mental health care processes. Previous attempts to incorporate technology into mental health interventions have been limited by their scope of data analysis and lack of specificity towards mental health applications. These prior systems have not addressed the need for a proactive strategy in mental health monitoring and intervention. The present invention overcomes these limitations by providing a holistic view of an individual's physiological state over time, offering a more accurate, predictive model of mental wellness that is actionable by mental health professionals. In doing so, it advances over the existing technologies, introducing a tool in addressing mental health issues and suicide rates among vulnerable populations.

The present invention relates to a software-based system that utilizes biometric data from wearable devices to facilitate a proactive approach to mental health management and suicide prevention. The system extracts and analyzes a wide array of physiological metrics collected by wearable technology, such as heart rate, sleep patterns, physical activity levels, and potentially dozens of other indicators that may have correlations with the mental state of an individual.

The collected biometric data is processed and analyzed using advanced algorithms to identify patterns, trends, or anomalies that could signify emerging mental health issues before they evolve into crises. The algorithms employed by the system are designed to detect subtle changes in an individual's physiological metrics over time, which may be indicative of deteriorating mental health or an increased risk of suicidal ideation.

The system's software component is responsible for the seamless integration of data from various wearable devices, ensuring compatibility and interoperability across different platforms and manufacturers. This allows for a comprehensive analysis of an individual's biometric data, regardless of the specific wearable device being used.

In addition to the data analysis capabilities, the system also incorporates a user interface that allows individuals to monitor their own mental health metrics and receive personalized recommendations for maintaining optimal mental well-being. This interface may include features such as data visualization tools, goal-setting functions, and access to mental health resources and support services.

The invention also includes a secure data storage and transmission infrastructure to ensure the privacy and confidentiality of sensitive personal information. This infrastructure employs industry-standard encryption protocols and access controls to safeguard user data from unauthorized access or breaches.

By leveraging the power of wearable technology and advanced data analytics, this invention provides a novel approach to mental health management and suicide prevention. The system's ability to detect early warning signs and provide timely interventions has the potential to significantly improve mental health outcomes and save lives.

### BRIEF DESCRIPTION OF THE DRAWINGS

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates a network architecture for systems and methods for predicting mental health crisis scenarios and initiating intervention in accordance with an exemplary embodiment of the invention.

FIG. 2 illustrates a system architecture for systems and methods for predicting mental health crisis scenarios and initiating intervention in accordance with an exemplary embodiment of the present invention.

FIG. 3 illustrates an exemplary computer implemented process for systems and methods for predicting mental health crisis scenarios and initiating intervention according to one embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

The detailed description set forth herein in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

### Network Architecture

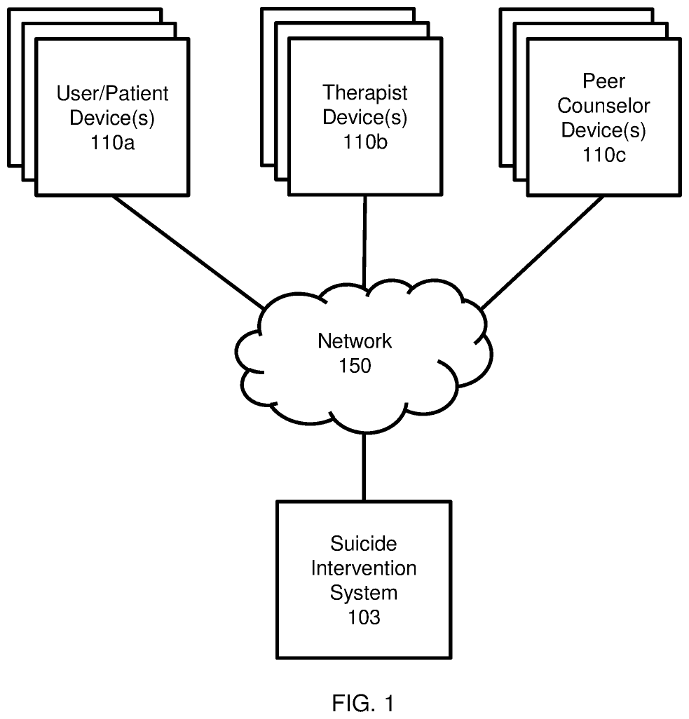

FIG. 1 illustrates an exemplary embodiment of systems and methods for predicting mental health crisis scenarios and initiating intervention according to one embodiment. FIG. 1 illustrates an exemplary embodiment of a system for patient interaction, monitoring and suicide intervention according to one embodiment. The system includes user/patient device(s) 110a, therapist device(s) 110b, peer counselor device(s) 110c, suicide intervention system 103, and a network 150 over which the various systems communicate and interact. The various components described herein are exemplary and for illustration purposes only and any combination or subcombination of the various components may be used as would be apparent to one of ordinary skill in the art. The system may be reorganized or consolidated, as understood by a person of ordinary skill in the art, to perform the same tasks on one or more other servers or computing devices without departing from the scope of the invention.

In one embodiment, the suicide intervention system 103 is a comprehensive software-based solution designed to proactively manage mental health and prevent suicide by leveraging biometric data from wearable devices and user input from various user devices, including patient devices 110a, therapist devices 110b, and peer counselor devices 110c. The system obtains and analyzes a wide range of data associated with lifestyle activities, such as sleep patterns, exercise habits, and heart rate, as well as mood-related information provided directly by the user.

The suicide intervention system 103 works by collecting and processing the biometric data and user input using advanced algorithms to identify patterns, trends, or anomalies that may indicate deteriorating mental health or an increased risk of suicidal ideation. By continuously monitoring and analyzing this data, the system can detect subtle changes in an individual's physiological metrics and mood over time, enabling early detection of potential mental health concerns before they escalate into crisis situations.

The system's algorithms are designed to adapt to each individual user's baseline metrics, establishing a personalized threshold for detecting deviations that may signify emerging mental health issues. When the system identifies patterns or anomalies that suggest a potential crisis, it triggers an intervention protocol, which may include alerting mental health professionals, such as therapists or peer counselors, and initiating communication with the user to provide timely support and assistance.

Alternatives to the suicide intervention system 103 may include manual monitoring of user data by mental health professionals, relying on self-reported information from users without the integration of biometric data, or using simplified algorithms that do not account for individual baselines and personalized thresholds. Other variations may involve utilizing different types of wearable devices or incorporating additional data sources, such as social media activity or environmental factors, to enhance the accuracy and effectiveness of the system's predictions and interventions.

In one embodiment, user device(s) 110, which include user/patient devices 110a, therapist devices 110b, and peer counselor devices 110c, are generally computers or computing devices with the functionality to communicate remotely over a network 150. These devices are operable to obtain data from users and provide data to other system components via network 150.

The user device(s) 110 serve as the primary interface for users, therapists, and peer counselors to interact with the system. They are designed to collect, send, and receive data, facilitating communication between all parties involved. For instance, user/patient devices 110a can gather and transmit biometric data from wearable devices, while therapist devices 110b and peer counselor devices 110c can receive this data for analysis and response.

The user device(s) 110 function by leveraging their built-in or connected hardware and software capabilities to communicate over network 150. This communication can be achieved through various protocols and technologies, such as Wi-Fi, cellular data, or wired connections. The devices can obtain data from users, such as biometric data or self-reported information, and transmit this data over the network 150 to other system components for processing and analysis. Similarly, these devices can receive data from other system components, such as analysis results or therapeutic recommendations, and present this data to the users.

Alternatives to the user device(s) 110 can include any computing device capable of network communication and data handling. These can range from desktop computers and laptops to smartphones and tablets. In some cases, specialized medical devices or dedicated hardware units could be used, particularly for collecting specific types of biometric data. The choice of device can depend on factors such as the specific needs of the user, the type of data being collected, and the preferred method of communication.

User device(s) 110 include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 150. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application obtains data from the network 150 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 421 or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to client/user devices or other devices in response to HTTP or other requests from client devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

### System Architecture

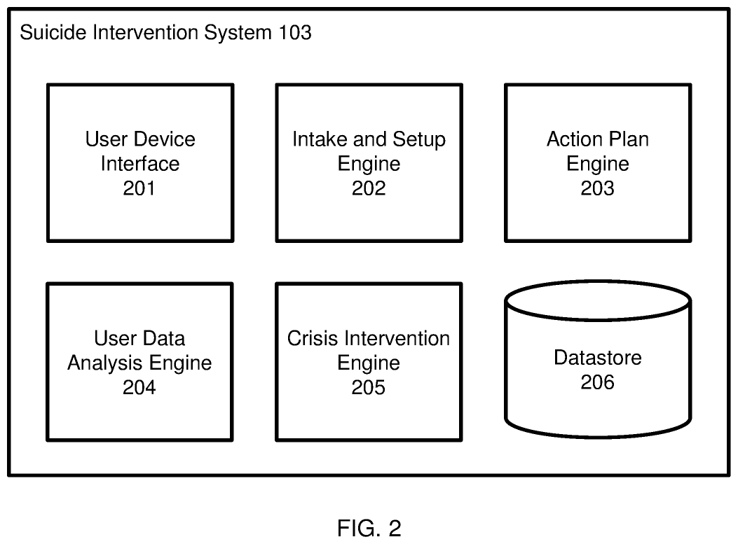

FIG. 2 illustrates an exemplary embodiment of the systems and methods for predicting mental health crisis scenarios and initiating intervention. FIG. 2 illustrates an exemplary embodiment of the suicide intervention system 103. The suicide intervention system 103 includes user device interface 201, intake and setup engine 202, action plan engine 203, user data analysis engine 204, crisis intervention engine 205, and datastore 206. The various components described herein are exemplary and for illustration purposes only and any combination or subcombination of the various components may be used as would be apparent to one of ordinary skill in the art. Other systems, interfaces, modules, engines, databases, and the like, may be used, as would be readily understood by a person of ordinary skill in the art, without departing from the scope of the invention. Any system, interface, module, engine, database, and the like may be divided into a plurality of such elements for achieving the same function without departing from the scope of the invention. Any system, interface, module, engine, database, and the like may be combined or consolidated into fewer of such elements for achieving the same function without departing from the scope of the invention. All functions of the components discussed herein may be initiated manually or may be automatically initiated when the criteria necessary to trigger action have been met.

In one embodiment, the user device interface 201 is a subsystem designed to communicate with user/patient devices, therapist devices, and peer support counselor devices, and to obtain data from these devices. The user device interface 201 serves as a conduit for data exchange between the different devices and the system.

The user device interface 201 obtains various types of data from the user/patient devices. This can include health and physiology data such as sleep data and heart rate data, mood data, and journal data. The interface 201 is also capable of obtaining action plan information from therapist user devices. This data collection allows the system to gather a comprehensive set of information about the user's mental and physical state, as well as the therapeutic strategies planned by the therapists.

The user device interface 201 operates by establishing communication links with the various devices. It can use different communication protocols and technologies, depending on the specific device it is interacting with. Once a communication link is established, the interface 201 can request and receive data from the devices, or send data to the devices. During a predicted crisis scenario, the interface 201 can provide communication to user devices and/or obtain communications from user devices, facilitating real-time response and intervention.

Alternatives to the user device interface 201 can include separate interfaces for each type of device, rather than a single interface for all devices. For instance, there could be a dedicated interface for user/patient devices, another for therapist devices, and another for peer support counselor devices. This could allow for more specialized communication and data handling for each type of device. Alternatively, the functionality of the user device interface 201 could be integrated directly into the devices themselves, eliminating the need for a separate interface. In one embodiment, the intake and setup engine 202 is a subsystem that manages risk assessment status and assigns risk status indicators to user/patient profiles. It serves as a central component in the initial setup and ongoing management of user profiles within the system.

The intake and setup engine 202 uses therapist assessment information and/or risk indication input to establish and associate a user profile with an appropriate risk status indication. This risk status serves as a key parameter in determining the level of attention and type of intervention required for a particular user. The risk status indication can be updated over time based on new assessment information or changes in the user's condition.

The intake and setup engine 202 operates by receiving and processing assessment information from therapists and other sources. This information can include various factors related to the user's mental health, such as their history, current symptoms, and any known risk factors. The engine 202 then uses this information to assign a risk status indicator to the user's profile. This indicator can be used by other parts of the system to determine the appropriate response if and when a crisis scenario is predicted.

Alternatives to the intake and setup engine 202 can include separate engines for intake and setup, rather than a single engine performing both functions. This could allow for more specialized processing and management of intake and setup tasks. Another alternative could be to integrate the functionality of the intake and setup engine 202 directly into the user device or therapist device, eliminating the need for a separate engine.

In one embodiment, the action plan engine 203 is a subsystem designed to manage and monitor a user's action plan, which is a set of goals and objectives related to their mental health treatment. At a high level, the action plan engine 203 is responsible for obtaining initial action plan data, updating the action plan as needed, and generating alerts and notifications based on the user's progress and adherence to the plan.

The action plan engine 203 works in conjunction with the user data analysis engine 204 to monitor the user's behavior characteristics and compare them to the established action plan. When the user data analysis engine 204 detects deviations from the action plan, the action plan engine 203 generates alerts to notify relevant parties, such as the user, their therapist, or designated support individuals. These alerts serve as a prompt for intervention or support to help the user stay on track with their mental health goals.

Additionally, the action plan engine 203 generates notifications to acknowledge and reinforce the user's progress when they achieve specific goals or objectives outlined in their action plan. These notifications serve as positive feedback and encouragement, helping to maintain the user's motivation and engagement in their mental health treatment.

The action plan engine 203 may obtain initial action plan data through various means, such as direct input from the user or their therapist, or by importing data from external sources. As the user progresses through their treatment, the action plan engine 203 can obtain updated action plan data to reflect changes in goals, objectives, or strategies based on the user's needs and progress.

Alternatives to the action plan engine 203 may include manual tracking and monitoring of the user's action plan by their therapist or support team, or using a simplified reminder system that sends periodic prompts to the user without considering their actual progress or behavior characteristics. Another variation could involve integrating the action plan engine 203 with a rewards system, where users earn points or badges for achieving their goals, providing an additional layer of motivation and engagement.

In one embodiment, the user data analysis engine 204 is a subsystem designed for the comprehensive analysis of user/patient data. Its primary function is to identify patterns and trends within the data, detect shifts that cross predefined thresholds, and leverage artificial intelligence/machine learning (AI/ML) technologies to enhance the analysis process.

The user data analysis engine 204 operates by first collecting user data, which may include biometric measures, mood assessments, and journal entries, among other types of data. It then applies AI/ML algorithms to this data to identify patterns and trends that may not be immediately apparent. These patterns and trends can indicate changes in the user's mental health status or predict potential crisis scenarios. By identifying shifts in data that cross certain thresholds, the engine can flag potential concerns that warrant closer attention or immediate intervention.

One of the key features of the user data analysis engine 204 is its ability to generate feedback data based on its analysis. This feedback is provided to users for the purpose of self-learning, enabling them to recognize behaviors or conditions that may require adjustment or regulation. This aspect of the engine supports proactive mental health management by empowering users with insights into their own health data.

Furthermore, the engine's predictive capabilities are a critical aspect of its functionality. By employing AI/ML in the analysis of user data, the engine is capable of predicting crisis circumstances before they occur. This predictive analysis is based on the identification of certain patterns, trends, and shifts in the data that are indicative of a crisis scenario.

Alternatives to the user data analysis engine 204 could involve different approaches to data analysis. For instance, instead of relying heavily on AI/ML, traditional statistical methods could be used to identify patterns and trends in the data. Additionally, the engine could be configured to focus on specific types of data, such as biometric data or mood data, depending on the primary concerns or needs of the user. Another alternative could involve a more interactive approach, where users provide direct feedback on the accuracy of the engine's predictions and analysis, which could be used to continually refine and improve the AI/ML algorithms.

In one embodiment, the crisis intervention engine 205 is a subsystem responsible for initiating and managing crisis intervention protocols when the user data analysis engine 204 determines that a user is experiencing a crisis scenario. The crisis intervention engine 205 operates at a high level by selecting and implementing an appropriate intervention protocol based on the user's assigned risk status.

The crisis intervention engine 205 works by assigning different intervention protocols to users based on their risk level. For instance, a first protocol may be implemented for lower-risk users, while a second protocol, which may involve more intensive intervention measures, is implemented for higher-risk users. When the user data analysis engine 204 predicts a crisis, the crisis intervention engine 205 triggers communication with the user/patient and/or their therapist, with the nature of the communication determined by the user's risk status assessment.

For lower-risk users, the communication may be a messaging prompt or text message, providing support and resources to help the user cope with the crisis. In contrast, for higher-risk users, the crisis intervention engine 205 may establish more urgent communication, such as initiating a phone call with the user/patient within a specified time frame. If communication with the user/patient cannot be established within the designated time frame, the crisis intervention engine 205 may trigger additional safety plan actions, such as notifying emergency contacts and/or emergency medical services, to ensure the user's safety and well-being.

Alternatives to the crisis intervention engine 205 may include a manual intervention process, where therapists or support staff monitor user data and initiate interventions based on their clinical judgment, without the use of automated risk assessment and protocol assignment. Another variation could involve a more granular risk assessment scale, with multiple intervention protocols tailored to specific risk levels, allowing for a more personalized approach to crisis intervention. Additionally, the crisis intervention engine 205 could be expanded to incorporate machine learning algorithms that continuously refine the intervention protocols based on user outcomes and feedback, optimizing the effectiveness of the crisis intervention process over time.

In one embodiment, Datastore 206 serves as a centralized repository for storing data generated and utilized by the various components of the suicide intervention system 103. This subsystem is designed to handle a wide range of data types, including but not limited to user biometric data, mood assessments, journal entries, therapist notes, and action plans.

Datastore 206 operates by receiving data from the system components, which it then organizes, secures, and stores for future access. The stored data can be retrieved for various purposes such as analysis by the user data analysis engine 204, generating user feedback, or updating user profiles. One of the key functions of Datastore 206 is to aggregate data over time, which supports longitudinal studies and research efforts aimed at understanding and improving suicide prevention strategies.

Furthermore, Datastore 206 plays a crucial role in the training and refinement of AI/ML algorithms within the system. By providing a comprehensive dataset that includes a wide array of user interactions, responses, and outcomes, the datastore enables machine learning models to learn from real-world cases, thereby improving their accuracy and effectiveness in predicting crisis scenarios and suggesting interventions.

Alternatives to Datastore 206 could involve distributed storage systems where data is stored across multiple locations or devices, rather than in a single centralized repository. This approach could offer advantages in terms of data redundancy and resilience. Another alternative could be the use of blockchain technology to ensure the integrity and security of the stored data, providing a tamper-proof record of all data transactions within the system. Additionally, cloud-based storage solutions could be employed to offer scalability and flexibility, accommodating the system's growing data storage needs over time.

### Computer Implemented Process

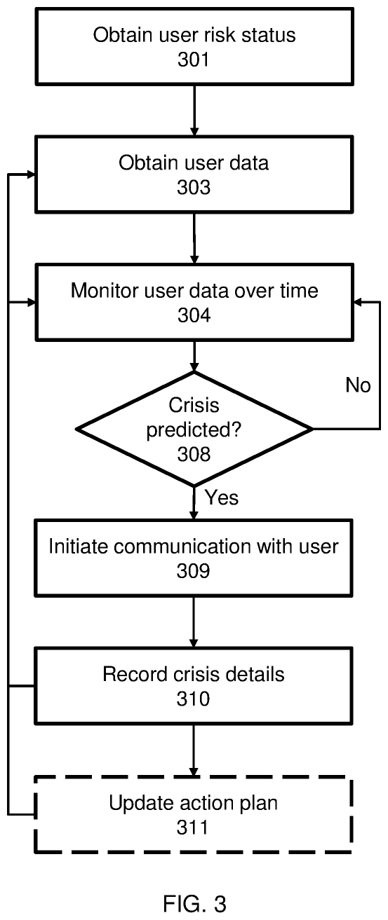

FIG. 3 illustrates an exemplary process for predicting crisis circumstances and initiating intervention according to one embodiment of the invention. The process comprises obtaining user risk status 301, obtaining initial user data 303, monitoring user data for progress 304, determining whether predictive crisis criteria have been met 308, initiating communication with the user 309, recording crisis details 310, and optionally updating an action plan 311. The process steps described herein may be performed in association with a system such as that described in FIG. 1 and/or FIG. 2 above or in association with a different system. The process may comprise additional steps, fewer steps, and/or a different order of steps without departing from the scope of the invention as would be apparent to one of ordinary skill in the art.

In one embodiment, the software process begins at step 301, which involves obtaining the user's risk status. The user, who may be a patient receiving treatment for mental health concerns associated with suicide risk, may undergo an assessment conducted by a therapist. This assessment can be performed during an intake appointment or consultation, where the therapist evaluates the user's mental health and suicide risk.

The process of obtaining the risk status involves assigning a risk status indicator to the user's profile. The risk status is categorized into multiple risk tiers, each representing a different level of expected risk for suicidal action by the user. In this embodiment, the risk tiers include low risk, medium or average risk, and high risk. However, the number of risk tiers can be modified to include fewer or more tiers without departing from the scope of the invention.

The risk status indicator is stored within the user's profile, allowing the software system to access and utilize this information for subsequent processes related to mental health treatment and suicide prevention. By categorizing users into different risk tiers, the system can adapt its approach and allocate resources accordingly, providing more responsive monitoring and support for users in higher risk categories.

Alternatives to this process may include using a numerical scale instead of descriptive risk tiers, incorporating additional factors such as the user's history or environmental circumstances into the risk assessment, or automating the risk assessment process using machine learning algorithms trained on historical data. Another variation could involve continuous monitoring and updating of the risk status based on real-time data from the user's wearable devices or self-reported information, rather than relying solely on periodic assessments by a therapist.

In one embodiment, at step 303, the software process involves obtaining initial user data. This data can include a variety of information such as sleep data, activity data, mood data, and journal data. The process is designed to capture this data at recurring intervals, which allows for the identification of changes over time. The frequency of data collection can vary, ranging from multiple times per day to weekly intervals.

The user data that the process obtains can be categorized into two main types: biometric data and mood data. Biometric data can be collected from a wearable device worn by the user or a user device such as a smartphone, tablet, or computing device. This data can include heart rate variability, cortisol levels, electroencephalography, galvanic skin response, pupil dilation, sleep patterns, physical activity levels, voice analysis, eye movement, blood pressure, and heart rate. Changes in these biometric measures can indicate changes in sleep status and/or mood.

Mood and journal data can be collected from a smartphone, tablet, or other computing device. Mood data can be obtained by presenting the user with questions throughout the day, with the responses recorded and tracked over time. Journal data can include written data, audio data, and voice-to-text data, providing further insight into the user's experiences and mood changes throughout the day.

The process may comprise obtaining, by a computing device, first biometric data from at least one wearable device associated with a user, the first biometric data comprising data associated with at least one of sleep, physical activity, and stress. The process may comprise obtaining, by a computing device, first psychological data from at least one user device, the first psychological data comprising data associated with a mood of the user.

The initial user data obtained by the process can be used to establish a baseline for biometrics and/or mood. This baseline can be updated or adjusted over time using a multi-day rolling baseline. This allows for a dynamic and responsive tracking of the user's condition. Establishing a baseline may comprise providing the first biometric data and first psychological data to at least one artificial intelligence algorithm, wherein the at least one artificial intelligence algorithm analyzes the first biometric data and first psychological data to establish at least one baseline metric for the user. This allows the process to be user-specific and thus sensitive to the key factors affecting a given user thereby increasing the likelihood of proactively predicting a user-specific crisis situation.

Establishing a baseline may comprise correlating biometric data with mood data. Establishing a baseline may comprise employing a machine learning algorithm which learns the relationship(s) between biometric data for an individual and mood for that individual. The machine learning model may be trained on subjective mood data and objective biometric data such that the model is able to identify or predict mood changes based on the biometric data or changes in the biometric data. The machine learning algorithm may continue to learn and adjust to individual biometrics and mood data as more data is obtained. The amount of change in biometric data may more significantly impact one individual as compared to another and the machine learning model may learn and account for these differences. For example, one individual may be more sensitive to mood changes with smaller biometric changes than another individual. Considering sleep as a biometric measure, for example, different individuals may experience different amounts of mood shift depending on the amount they sleep on a given night or over a series of nights. For example, one individual may experience negative mood changes when getting less than 7 hours of sleep a night while another person may experience negative mood changes when getting less than 5 hours of sleep a night. Sleep is merely one example of a biometric measure which may be correlated to mood and other metrics may be used without departing from the scope of the invention.

The machine learning model is operable to learn and account for these individual-specific relationship(s) between biometrics and mood. The model may determine at least one biometric data threshold that, if exceeded or not met, is indicative of a likely mood shift. In this way, even in the absence of accurate subjective data (e.g. self-reported mood), the model may predict a potential crisis scenario based on biometric data. In one aspect, the threshold(s) may serve as indicators of an opportunity for early intervention in an attempt to prevent progression to a more extreme mood change. For example, a subtle reminder or notification may be provided to a user when they are not hitting a first sleep duration threshold (e.g. a minimum target sleep duration to maintain a positive mood or a stable or improving mood trend), while a more aggressive intervention may be triggered when sleep duration falls below a second sleep duration lower than the first sleep duration threshold (e.g. a minimum target sleep duration to avoid a negative mood shift).

Alternatives to this software process could involve different methods of data collection. For instance, biometric data could be collected using medical devices or specialized sensors, rather than wearable devices or user devices. Mood data could be collected through interviews or surveys, rather than through prompts on a user device. The frequency and method of data collection could also be adjusted based on the specific needs and circumstances of the user.

In one embodiment, at step 304, the software process involves monitoring user data over time. This includes obtaining ongoing user data and analyzing it to identify trends and changes. These trends and changes can be related to aspects of the user's action plan. They can be computed relative to a baseline or typical status for the user, or reflect periodic changes, such as day to day or week to week changes. The data associated with monitoring may be obtained at a time that is later than the initial user data or the data used in establishing a baseline for the user.

Monitoring may comprise obtaining, by a computing device, second biometric data from at least one wearable device associated with a user, the second biometric data comprising data associated with at least one of sleep, physical activity, and stress. Monitoring may comprise obtaining, by a computing device, second psychological data from at least one user device, the second psychological data comprising data associated with a mood of the user. The obtained data may be provided to at least one artificial intelligence algorithm, wherein the at least one artificial intelligence algorithm analyzes the second biometric data and second psychological to identify patterns, trends, or anomalies indicative of emerging mental health issues. The identifying of patterns, trends, or anomalies may comprise comparing with the at least one baseline metric to determine an amount of change or deviation relative to the at least one baseline metric.

Monitoring user data for progress involves identifying a change or shift in the data. This change or shift is compared against a threshold indicative of a potential concern. The threshold is determined based on the user's risk status. For instance, smaller deviations from normal may be used as thresholds for higher risk patients, while larger deviations may be used for lower risk patients. A deviation of 30% may be used as the threshold in higher risk patients, while a deviation of 50% or greater may be used for lower risk patients. These percentages serve as examples, and additional thresholds may be used.

The process also includes monitoring user data for risk indicators. These risk indicators are behavioral characteristics that exceed or fail to meet certain criteria or thresholds. For instance, a risk indication may be triggered if sleep duration falls below a threshold level for several consecutive days, or if total sleep over several days falls below a threshold level. Another risk indicator trigger may be a sudden change in mood from one data point to another.

Monitoring user data for risk indicators can involve the use of artificial intelligence (AI) and machine learning. AI algorithms may be trained using aggregated data across multiple users or individuals with or without mental health issues. They may also be trained using user-specific data, making the algorithm unique to an individual. These AI algorithms can receive biometric, mood, and/or action plan progress information as input and analyze this information to predict a crisis. In some cases, monitoring user data for risk indicators can involve analyzing mood data and journal data using AI language processing algorithms such as natural language processing models and large language models.

Alternatives to this software process could involve different methods of data monitoring and analysis. For instance, rather than using AI and machine learning, traditional statistical methods could be used to identify trends and changes in the data. The thresholds used to trigger risk indications could also be adjusted based on different criteria. The type of data collected and analyzed could also vary, depending on the specific needs and circumstances of the user.

At step 308, the process determines if a crisis situation is predicted based on the monitoring performed in step 304. This determination is made by analyzing the user data collected in the previous steps, looking for patterns, trends, or anomalies that may indicate a potential crisis. The analysis may involve various artificial intelligence (AI) techniques, such as machine learning algorithms, which can be trained on historical data to identify risk indicators and predict crisis situations.

The AI analysis may take into account multiple factors, such as changes in the user's biometric data (e.g., heart rate, sleep patterns), mood data (e.g., responses to questionnaires), and behavioral data (e.g., social media activity, phone usage). By combining and analyzing these different types of data, the AI algorithms can build a comprehensive picture of the user's mental health status and detect subtle signs of deterioration that may not be apparent to human observers.

One approach to predicting a crisis situation is to establish a baseline for the user's normal behavior and mental health status, based on data collected over a period of time. This baseline can then be used as a reference point to detect deviations or anomalies in the user's data. For example, if the user's sleep patterns or mood scores start to deviate significantly from their baseline, this may indicate a potential crisis situation.

The amount of deviation required to trigger a crisis prediction may be determined by a threshold value, which can be set based on various factors, such as one or more of the user's individual characteristics, medical history, risk profile, and risk status indicator. The threshold may be adjusted over time as the system learns more about the user's behavior and mental health patterns. For example, a deviation or change of 30% or more may be used as the threshold in higher risk patients, while a deviation or change of 50% or greater may be used for lower risk patients. These percentages serve as examples, and different and/or additional thresholds may be used without departing from the scope of the invention.

Alternatively, the crisis prediction may be based on specific risk indicators or patterns identified in the user's data, such as a sudden increase in negative sentiment in their journal data and/or social media posts, and/or a combination of biometric and behavioral changes that have been associated with crisis situations in the past. These risk indicators may be identified through machine learning techniques, such as decision trees, random forests, or deep learning neural networks, which can learn to recognize complex patterns in the data.

If a crisis situation is predicted, the process triggers an intervention protocol at step 308, which may involve initiating a process(es) to at least one of alerting the user, their support network, and/or mental health professionals, and providing resources and/or support to help the user manage the crisis. If no crisis is predicted, the process continues monitoring user data for risk indicators as new data is continually obtained at step 303, allowing for ongoing assessment and early prediction or detection of potential crisis situations.

At step 309, the process initiates communication with the user when a crisis scenario is predicted. The communication aims to establish contact with the user and provide support or intervention as needed. The means of communication may vary depending on the user's risk status indicator and the severity of the predicted crisis.

The process may employ a tiered response system based on the user's risk status. For users with a lower risk status, the communication may involve less intrusive methods, such as displaying a prompt on the user's device through a dedicated application or sending a text message. These methods allow the user to acknowledge the communication and provide a response at their own pace. For users with a higher risk status, the process may initiate more direct and immediate forms of communication, such as placing a phone call to the user. This ensures that the user receives prompt attention and support in case of a more severe crisis.

The specific communication method may also be determined by the degree of deviation from the user's baseline metrics. For example, if the user's data shows a 30% or greater change from their baseline, the process may generate a user prompt or send a text message to the user's device. However, if the deviation is more significant, such as a 50% or greater change, the process may establish audio or video communication with the user, such as through a phone call or video call. This allows for a more direct and personal interaction with the user, which may be necessary for addressing more serious crisis situations.

The process may set a maximum allowable time period for establishing communication with the user, which can vary based on the user's risk status. For example, the process may require establishing contact within 5 minutes for high-risk users and within 10 minutes for lower-risk users. The communication may also require a response from the user, such as acknowledging a prompt, answering a question, replying to a text message, or answering a phone call. If no response is received within the specified time frame, the process may trigger the activation of a safety plan, which can include alerting emergency contacts, notifying emergency medical services, obtaining the user's geolocation information from their device, and sharing this information with the appropriate entities involved in the safety plan.

In addition to communicating with the user, the process may also alert the user's therapist and/or peer support counselor when a crisis is predicted. This can be done by sending a prompt or text message to the therapist and/or counselor's device simultaneously or at substantially the same time as the user is contacted. The alert may indicate that a phone or video call to the user is required or recommended, depending on the severity of the situation. The therapist and/or counselor may then initiate the phone call to the user, providing professional support and guidance. In cases where the therapist or counselor is unavailable to make the call, the process may alert alternate support personnel, such as crisis intervention hotline staff, to contact the user instead.

Alternatively, the process may employ a more automated approach to communication, using chatbots or virtual assistants to initiate contact with the user. These AI-powered tools can engage the user in conversation, assess their emotional state, and provide initial support or guidance. The chatbots can be programmed with crisis intervention protocols and can escalate the interaction to human support staff if necessary. This approach may be useful for handling a large volume of users or for providing 24/7 support coverage.

At step 310, the process records crisis details, which involves capturing and storing relevant information about the predicted crisis situation and the subsequent intervention efforts. This step is crucial for documenting the crisis event, facilitating later analysis and review, and improving the accuracy of future crisis predictions.

The process identifies and collects the user data that is most relevant to the crisis situation, such as the specific biometric readings, mood scores, or behavioral patterns that triggered the crisis prediction. This data may be flagged, highlighted, or otherwise associated with the crisis event to make it easily retrievable for future reference. The collected data is then securely stored in a database, along with a timestamp and any other relevant metadata, such as the user's risk status or the specific intervention protocol that was initiated.

The recorded user data serves multiple purposes. Firstly, it can be used as training data to improve the accuracy and performance of the predictive crisis models. By analyzing the data associated with confirmed crisis events, the machine learning algorithms can learn to better identify patterns and risk indicators, reducing false positives and false negatives in future predictions. The models can be continuously updated and refined as new crisis data becomes available, ensuring that the system remains effective and relevant over time.

Secondly, the recorded user data can be provided to the user's therapist or mental health professional for review and analysis. This allows the therapist to gain a more comprehensive understanding of the user's mental health status and the specific circumstances that led to the crisis situation. The therapist can use this information to adjust the user's treatment plan, prescribe new interventions or coping strategies, or schedule additional sessions to address the underlying issues. By having access to detailed crisis data, therapists can provide more targeted and effective care to their patients.

In addition to user data, the crisis details may also include information about the interaction (or lack thereof) with the user during the intervention process. This may include details such as whether communication was successfully established with the user, how the user responded to the communication attempts, and any specific actions taken by the support team. For example, if the user acknowledged a prompt and indicated that they were not in crisis, this information would be recorded for future reference. Similarly, if the user did not respond to multiple communication attempts, this lack of interaction would also be noted in the crisis details.

Alternatively, the process may involve recording additional types of information, such as the duration of the crisis event, the specific support personnel involved in the intervention, or any external factors that may have contributed to the crisis (e.g., a recent life stressor or triggering event). This information can provide valuable context for understanding the crisis situation and informing future prevention and intervention strategies.

The recorded crisis details may also be used for quality assurance and accountability purposes. By maintaining a detailed record of each crisis event, the system can demonstrate that appropriate actions were taken in accordance with established protocols and best practices. This can be important for legal and regulatory compliance, as well as for building trust with users and their support networks.

The process of updating the action plan begins with a thorough review of the user's current plan and the circumstances surrounding the predicted crisis. If the crisis prediction was accurate and not a false positive, it suggests that the existing action plan may need to be revised to better address the user's needs. The process analyzes the factors that may have contributed to the crisis, such as triggers, hindrances, or other negative influences, and incorporates this information into the updated plan.

The updated action plan may include a wide range of recommendations tailored to the individual user. These recommendations may consist of adjustments to the user's medication regimen, such as changing dosages, adding new medications, or discontinuing ineffective ones. The plan may also include suggestions for improving sleep habits, such as establishing a consistent sleep schedule, creating a relaxing bedtime routine, or avoiding stimulating activities before bed.

Diet and exercise recommendations may also be incorporated into the updated action plan. The process may suggest specific dietary changes, such as increasing intake of nutrient-rich foods, reducing consumption of processed or sugary foods, or avoiding substances that may negatively impact mental health, such as alcohol or caffeine. Exercise recommendations may include specific types of physical activity, duration, and frequency, based on the user's abilities and preferences.

The updated action plan may also address the user's social support system and therapeutic interventions. This may include recommendations for increasing the frequency of therapy or counseling sessions, attending support groups, or engaging in activities that promote social connection and emotional well-being. The plan may also identify specific help and support resources, such as crisis hotlines, online communities, or local mental health services, that the user can access when needed.

In addition to these recommendations, the updated action plan may include specific goals and objectives for the user to work towards. These goals may be related to symptom reduction, improved functioning, or increased resilience, and may be broken down into smaller, achievable steps. The process may also incorporate feedback from the user and their support team, such as their therapist or peer counselor, to ensure that the plan is realistic and aligned with the user's values and preferences.

Alternatively, the process may utilize machine learning algorithms to generate personalized action plan recommendations based on the user's unique characteristics and history. These algorithms can analyze large datasets of user information, including demographic data, clinical histories, and treatment outcomes, to identify patterns and predict which interventions are most likely to be effective for a given individual. This approach may allow for more precise and targeted recommendations, reducing the need for trial and error in finding the optimal treatment strategy.

The updated action plan is then communicated to the user and their support team, who can review and discuss the recommendations and make any necessary adjustments. The plan is regularly monitored and evaluated to assess its effectiveness in preventing future crisis scenarios and promoting the user's overall mental health and well-being. As the user's needs and circumstances change over time, the action plan may be further modified to ensure that it remains relevant and effective.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

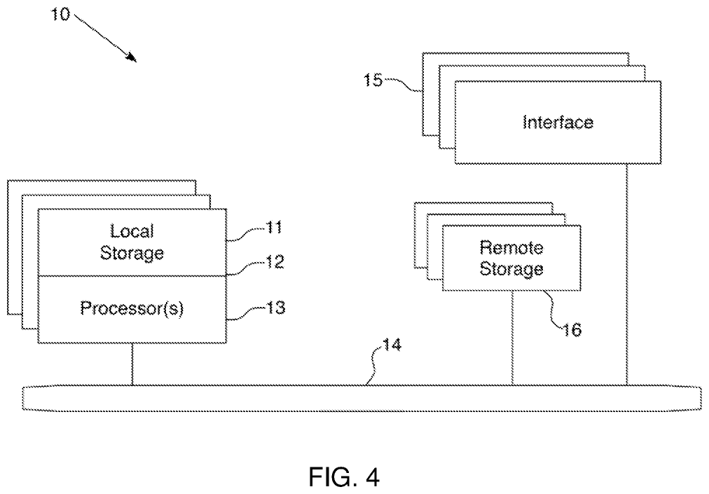

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, controllers, components, process steps or the like may be and/or comprise hardware and/or software as described herein. For example, the systems, engines, and subcomponents described herein may be and/or comprise computing hardware and/or software as described herein in association with FIGS. 4-7. Furthermore, any of the above mentioned systems, units, modules, engines, controllers, components, interfaces or the like may use and/or comprise an application programming interface (API) for communicating with other systems units, modules, engines, controllers, components, interfaces or the like for obtaining and/or providing data or information.

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

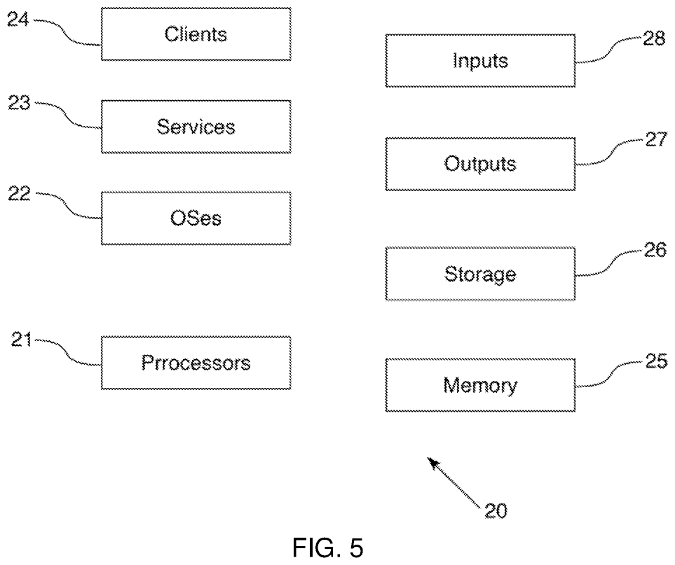

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

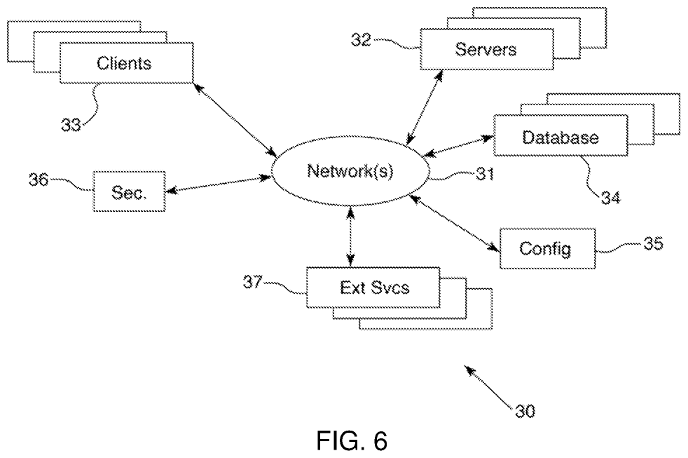

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications are implemented on a smartphone or other electronic device, client applications may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

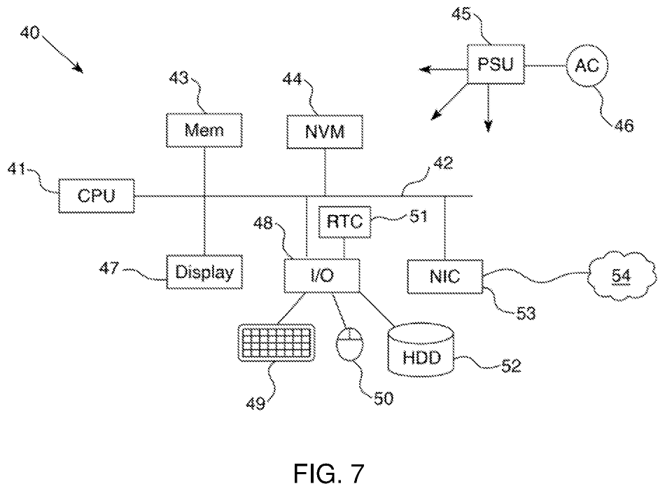

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and/or a process associated with the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A method for facilitating mental health management and suicide prevention, comprising:
obtaining, by a computing device, a risk status indicator associated with a user, the risk status indicator indicative of a likelihood the user may experience a mental health crisis;
obtaining, by a computing device, first biometric data from at least one wearable device associated with a user, the first biometric data comprising data associated with at least one of sleep, physical activity, and stress;
obtaining, by a computing device, first psychological data from at least one user device, the first psychological data comprising data associated with a mood of the user;
providing the first biometric data and first psychological data to at least one artificial intelligence algorithm, wherein the at least one artificial intelligence algorithm analyzes the first biometric data and first psychological data to establish at least one baseline metric for the user;
obtaining, by a computing device, second biometric data from at least one wearable device associated with a user, the second biometric data comprising data associated with at least one of sleep, physical activity, and stress;
obtaining, by a computing device, second psychological data from at least one user device, the second psychological data comprising data associated with a mood of the user;
providing the second biometric data and second psychological data to at least one artificial intelligence algorithm, wherein the at least one artificial intelligence algorithm analyzes the second biometric data and second psychological to identify patterns, trends, or anomalies indicative of emerging mental health issues, wherein identifying comprises comparing with the at least one baseline metric to determine an amount of change or deviation relative to the at least one baseline metric;
determining, based on the analysis, whether a crisis situation is predicted, the determining comprising determining that the amount of change or deviation exceeds a threshold, the threshold defined based on the risk status indicator; and
initiating, by the computing device, a communication with the user when a crisis situation is predicted, wherein the form of communication initiated is based on at least one of the risk status indicator and the amount of change or deviation.

2. The method of claim 1, wherein the biometric data comprises at least one of heart rate variability (HRV), cortisol levels, electroencephalography (EEG), galvanic skin response (GSR), pupil dilation, sleep patterns, physical activity levels, voice analysis, eye movement, blood pressure, and heart rate.

3. The method of claim 1, wherein the data associated with a mood of the user is obtained from one or more questions presented to the user at periodic intervals.

4. The method of claim 3, further comprising analyzing the mood data in conjunction with the biometric data to identify patterns, trends, or anomalies indicative of emerging mental health issues.

5. The method of claim 1, wherein the data associated with a mood of the user comprises obtaining journal data from the user via a user device, wherein the journal data comprises at least one of written data, audio data, and voice-to-text data.

6. The method of claim 5, further comprising analyzing the journal data using artificial intelligence language processing algorithms to identify risk indicators.

7. The method of claim 1, further comprising updating the baseline data over time using a multi-day rolling baseline.

8. The method of claim 1, wherein initiating a communication with the user comprises at least one of generating a prompt on a user device, sending a text message to the user, and establishing audio and/or video communication with the user.

9. The method of claim 1, wherein the at least one artificial intelligence algorithm is operable to identify user-specific triggers or key factors affecting a mood of the individual.

10. The method of claim 9, wherein determining whether a crisis situation is predicted comprises evaluating the user-specific triggers or key factors affecting a mood of the individual.

11. The method of claim 1, further comprising alerting at least one of a therapist and a peer support counselor associated with the user when a crisis situation is predicted.

12. The method of claim 1, further comprising recording crisis details when a crisis situation is predicted, wherein the crisis details comprise user data associated with the crisis and interaction information with the user.

13. The method of claim 1, further comprising updating an action plan associated with the user when a crisis situation is predicted, wherein the action plan comprises recommendations for improving or maintaining mental health status.

14. A system for facilitating mental health management and suicide prevention, comprising:
a computing device;
at least one wearable device associated with a user; and
a software application executing on the computing device, the software application configured to:
obtain, by a computing device, a risk status indicator associated with a user, the risk status indicator indicative of a likelihood the user may experience a mental health crisis;
obtain, by a computing device, first biometric data from at least one wearable device associated with a user, the first biometric data comprising data associated with at least one of sleep, physical activity, and stress;
obtain, by a computing device, first psychological data from at least one user device, the first psychological data comprising data associated with a mood of the user;
provide the first biometric data and first psychological data to at least one artificial intelligence algorithm, wherein the at least one artificial intelligence algorithm analyzes the first biometric data and first psychological data to establish at least one baseline metric for the user;
obtain, by a computing device, second biometric data from at least one wearable device associated with a user, the second biometric data comprising data associated with at least one of sleep, physical activity, and stress;
obtain, by a computing device, second psychological data from at least one user device, the second psychological data comprising data associated with a mood of the user;
provide the second biometric data and second psychological data to at least one artificial intelligence algorithm, wherein the at least one artificial intelligence algorithm analyzes the second biometric data and second psychological to identify patterns, trends, or anomalies indicative of emerging mental health issues, wherein identifying comprises comparing with the at least one baseline metric to determine an amount of change or deviation relative to the at least one baseline metric;
determine, based on the analysis, whether a crisis situation is predicted, the determining comprising determining that the amount of change or deviation exceeds a threshold, the threshold defined based on the risk status indicator; and
initiate, by the computing device, a communication with the user when a crisis situation is predicted, wherein the form of communication initiated is based on at least one of the risk status indicator and the amount of change or deviation.

15. The system of claim 14, wherein the at least one wearable device comprises at least one of a smartwatch and a smartphone.

16. The system of claim 14, wherein the software application is further configured to obtain mood data and journal data from the user via a user device.

17. The system of claim 14, wherein the software application is further configured to analyze the mood data and journal data in conjunction with the biometric data to identify patterns, trends, or anomalies indicative of emerging mental health issues.

18. The system of claim 14, wherein the software application is further configured to update the baseline over time using a multi-day rolling baseline.

19. The system of claim 14, wherein the software application is further configured to initiate a communication with the user by at least one of generating a prompt on a user device, sending a text message to the user, and establishing audio or video communication with the user.

20. The system of claim 14, wherein the at least one artificial intelligence algorithm is operable to identify user-specific triggers or key factors affecting a mood of the individual, and wherein determining whether a crisis situation is predicted comprises evaluating the user-specific triggers or key factors affecting a mood of the individual.
